(19) United States
(12) Patent Application Publication
(10) Pub. No.: US 2017/0174725 A1
(43) Pub. Date: Jun. 22, 2017

(54) PROCESSES FOR THE PREPARATION OF OXYTOCIN ANALOGUES

(71) Applicant: Hoffmann-La Roche Inc., Little Falls, NJ (US)

(72) Inventors: Konrad Bleicher, Freiburg (DE); Anton Cueni, Brislach (CH); Kurt Puentener, Ueken (CH); Junichi Shiina, Kamakura (JP)

(73) Assignee: Hoffmann-La Roche Inc., Little Falls, NJ (US)

(21) Appl. No.: 15/426,550

(22) Filed: Feb. 7, 2017

Related U.S. Application Data
(63) Continuation of application No. PCT/EP2015/067881, filed on Aug. 4, 2015.

(30) Foreign Application Priority Data
Aug. 7, 2014 (EP) 14180161.3

(51) Int. Cl. C07K 7/54

(52) U.S. Cl. CPC C07K 7/54

(57) ABSTRACT

Provided herein is a process for the preparation of oxytocin receptor agonists that have the potential to be used for the treatment of neurological disorders.

### CROSS REFERENCE TO RELATED APPLICATIONS

This application is a continuation of International Application No. PCT/EP2015/067881, having a filing date of 4 Aug. 2015, which claims benefit under 35 U.S.C. 119 to European Patent Application No. 14180161.3, filed 7 Aug. 2014, the entire contents of each of which are incorporated herein by reference.

The invention relates to a new process for the preparation of Oxytocin analogues of formula I

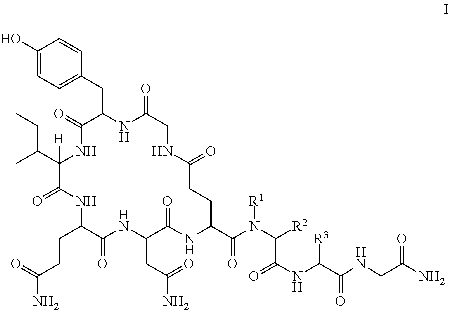

wherein
R¹ is hydrogen or C₁₋₇-alkyl and
R² is hydrogen or C₁₋₇-alkyl; or
R¹ and R² together with the nitrogen and the carbon atom to which they are attached form a 5-membered heterocyle which is optionally substituted with hydroxy or halogen;
R³ is C₁₋₇-alkyl

and its corresponding enantiomers and/or optical isomers thereof.

Oxytocin analogues of the formula I act as oxytocin receptor agonists and have the potential to be used for the treatment of neurological disorders such as autism, stress, including post-traumatic stress disorder, anxiety, including anxiety disorders and depression, schizophrenia, psychiatric disorders and memory loss, alcohol withdrawal, drug addiction and for the treatment of the Prader-Willi Syndrome (PCT Publication WO 2014/095773).

The preparation of the oxytocin analogues according to process described in the PCT Publication WO 2014/095773 is characterized by the following steps:

x¹) cleavage of Fmoc from a resin bound peptide precursor of the formula X

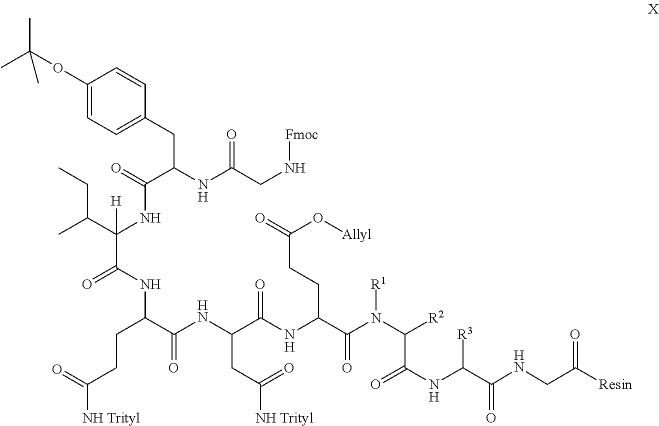

x²) cleavage of the allyl group in a subsequent step

x³) ring cyclization on the resin

x⁴) global deprotection and cleavage from the resin

x⁵) purification and isolation.

It was found that this process known in the art suffers from low overall yields and product selectivity.

Object of the present invention therefore was to improve the synthesis regarding yield and selectivity of the desired Oxytocin analogues.

The object could be achieved with the process of the present invention as outlined hereinafter below.

The process for the preparation of Oxytocin analogues of the formula I

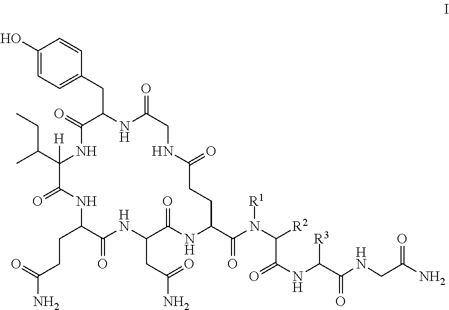

wherein
R¹ is hydrogen or C₁₋₇-alkyl and
R² is hydrogen or C₁₋₇-alkyl; or
R¹ and R² together with the nitrogen and the carbon atom to which they are attached form a 5-membered heterocyle which is optionally substituted with hydroxy or halogen;
R³ is C₁₋₇-alkyl

and of its corresponding enantiomers and/or optical isomers thereof comprises treating a resin bound peptide precursor of the formula II

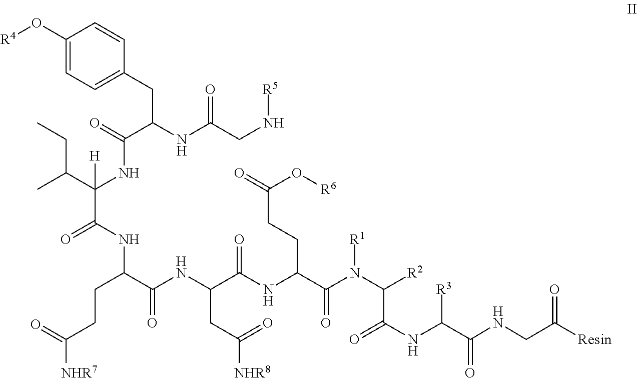

wherein
R¹, R² and R³ are as above and
R⁴ is a hydroxy protecting group;
R⁵ is Fmoc;
R⁶ is allyl, t-butyl, 1-adamantyl, 4-{N-[1-(4,4-dimethyl-2,6-dioxocyclohexylidene)-3-methylbutyl]amino}benzyl or phenylisopropyl;
R⁷ is an amide protecting group; and
R⁸ is an amide protecting group

and its corresponding enantiomers and/ or optical isomers thereof,

either according to the method:

a) wherein in case of R⁶ being allyl or 4-{N-[1-(4,4-dimethyl-2,6-dioxocyclohexylidene)-3-methylbutyl]amino}benzyl
a¹) the allyl group or the 4-{N-[1-(4,4-dimethyl-2,6-dioxocyclohexylidene)-3-methylbutyl]amino}benzyl group R⁶ is cleaved, in a subsequent step
a²) the Fmoc group R⁵ is cleaved, thereafter
a³) ring cyclization is effected on the resin, in a further step
a⁴) global deprotection and cleavage from the resin is effected, and optionally
a⁵) the oxytocin analogue of formula I so obtained is purified and isolated; or according to the method:

b) wherein in case of R⁶ being t-butyl, 1-adamantyl or phenylisopropyl;
b¹) the Fmoc group R⁵ is cleaved, thereafter
b²) global deprotection and cleavage from the resin is effected, in a further step
b³) ring cyclization is effected in solution, then optionally
b⁴) the oxytocin analogue of formula I so obtained is isolated and purified.

The following definitions are set forth to illustrate and define the meaning and scope of the various terms used to describe the invention herein.

The term “C₁₋₇-alkyl” relates to a branched or straight-chain monovalent saturated aliphatic hydrocarbon radical of one to seven carbon atoms, preferably one to four, more preferably one to two carbon atoms. This term is further exemplified by radicals as methyl, ethyl, n-propyl, i-propyl, n-butyl, s-butyl, i-butyl, or t-butyl, pentyl and its isomers, hexyl and its isomers and heptyl and its isomers.

Likewise the term “C₁₋₄-alkyl” relates to a branched or straight-chain monovalent saturated aliphatic hydrocarbon radical of one to four carbon atoms, with the preferences and the respective examples mentioned above.

The term “C₁₋₄-alkyloxy” relates to C₁₋₄-alkyl chain attached to an oxygen atom. This term is further exemplified by radicals as methoxy, ethoxy, n-propoxy, i-propoxy, n-butoxy, i-butoxy and t-butoxy.

The term “C₁₋₄-alkyloxycarbonyl” relates to a C₁₋₄-alkoxy chain attached to a carbonyl group and is further exemplified by the particular alkoxy radicals outlined above attached to a carbonyl group.

The term “C₂₋₄-alkenyl” relates to an unsaturated straight- or branched-carbon chain containing from 2 to 4 carbon atoms containing at least one double bond. This term is further exemplified by radicals as vinyl, allyl and butenyl and its isomers.

The term “halogen” refers to fluorine, chlorine, bromine or iodine.

The term “5-membered heterocyle” which is formed together with R¹ and R² with the nitrogen and the carbon atom to which they are attached stands for a pyrrolidine ring optionally substituted with hydroxy or halogen, particularly for the pyrrolidine ring of proline which is substituted by hydroxy or fluorine.

The term “amide protecting group” refers to an acid or Lewis acid sensitive substituent conventionally used to hinder the reactivity of the amide group. Suitable acid or Lewis acid sensitive amide protecting groups are described in Isidro-Llobet A., Alvarez, M. and Albericio F., “Amino Acid-Protecting Groups”, Chem. Rev. 2009, 109, 2455-2504., Chan W. C. and White P. D. “Fmoc Solid Phase Peptide Synthesis”, Oxford University Press and Green T., “Protective Groups in Organic Synthesis”, 4^{th} Ed. by Wiley Interscience, 2007, Chapter 7, 696 ff. Suitable amide protecting groups can therefore be selected from trityl, Tmob (2,4,6-trimethoxybenzyl), Xan (9-xanthenyl), Cpd (cyclopropyldimethylcarbinyl), Mbh (4,4′-dimethoxybenzhydryl) or Mtt (4-methyltrityl),

The term “hydroxy protecting group” used for substituent R⁴ refers to any substituents conventionally used to hinder the reactivity of the hydroxy group. Suitable hydroxy protecting groups are described in Isidro-Llobet A., Alvarez, M. and Albericio F., “Amino Acid-Protecting Groups”, Chem. Rev. 2009, 109, 2455-2504., Chan W. C. and White P. D. “Fmoc Solid Phase Peptide Synthesis”, Oxford University Press, Green T., “Protective Groups in Organic Synthesis”, Chapter 1, John Wiley and Sons, Inc.,1991, 10-142 and can be selected from C₁₋₄-alkyl which is optionally substituted with phenyl or halogenated phenyl; C₂₋₄-alkenyl; silyl which is optionally substituted with C₁₋₄-alkyl or phenyl or C₁₋₄-alkyloxycarbonyl.

The spiral bond “ [symbol] ”

stands for “ [symbol] ” or for “ [symbol] ” thus indicating chirality of the molecule.

Whenever a chiral carbon is present in a chemical structure, it is intended that all stereoisomers associated with that chiral carbon are encompassed by the structure as pure stereoisomers as well as mixtures thereof.

In a particular embodiment of the present invention the Oxytocin analogues have the formula Ia

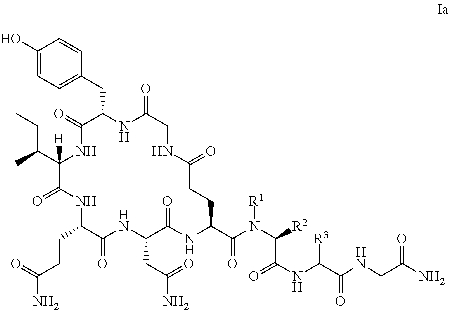

wherein R¹, R² and R³are as above.

R¹ is particularly hydrogen or C₁₋₄-alkyl, more particularly hydrogen or methyl.

R² is particularly hydrogen or C₁₋₄-alkyl, more particularly hydrogen.

R¹ and R² together with the nitrogen and the carbon atom to which they are attached particularly form the pyrrolidine ring of proline which is optionally substituted with hydroxy or halogen, particularly with hydroxy or fluorine.;

R³ particularly stands for n-butyl or i-butyl;

Even more particular Oxytocin analogues are listed below:

c[Gly-Tyr-Ile-Gln-Asn-Glu]-Gly-Leu-Gly-NH₂ (1)

c[Gly-Tyr-Ile-Gln-Asn-Glu]-Pro-Leu-Gly-NH₂ (2)

c[Gly-Tyr-Ile-Gln-Asn-Glu]-Sar-Leu-Gly-NH₂ (3)

c[Gly-Tyr-Ile-Gln-Asn-Glu]-Sar-Nle-Gly-NH₂ (4)

c[Gly-Tyr-Ile-Gln-Asn-Glu]-trans-4-fluoro-Pro-Leu-Gly-NH₂ (5)

c[Gly-Tyr-Ile-Gln-Asn-Glu]-trans-4-hydroxy-Pro-Leu-Gly-NH₂ (6).

The resin bound peptide precursor of the formula II has the formula

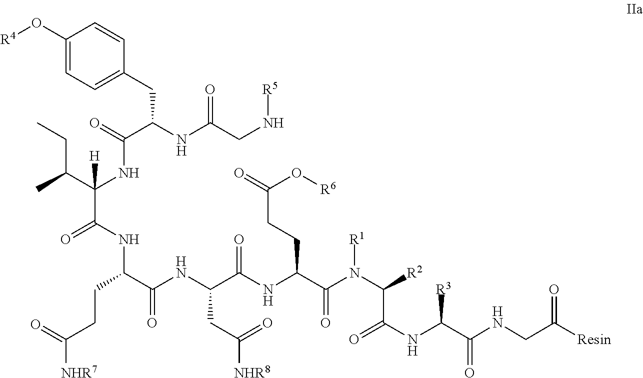

wherein R¹, R², R³, R⁴, R⁵, R⁶, R⁷ and R⁸ are as above.

R¹ is particularly hydrogen or C₁₋₄-alkyl, more particularly hydrogen or methyl.

R² is particularly hydrogen or C₁₋₄-alkyl, more particularly hydrogen.

R¹ and R² together with the nitrogen and the carbon atom to which they are attached particularly form the pyrrolidine ring of proline which is optionally substituted with hydroxy or halogen, particularly with hydroxy or fluorine. ;

R³ particularly stands for n-butyl or i-butyl;

R⁴ particularly is t-butyl, allyl, trityl, 2-chlorotrityl, t-butyloxycarbonyl, t-butyldiphenylsilyl or t-butyldimethylsilyl, but more particularly t-butyl;

R⁵ is Fmoc;

R⁶ particularly is allyl 1-adamantyl, 4-{N-[1-(4,4-dimethyl-2,6-dioxocyclohexylidene)-3-methylbutyl]amino}benzyl, phenylisopropyl or t-butyl, but more particularly allyl;

R⁷ particularly is trityl, 2-chlorotrityl, 4-methyltrityl, but more particularly trityl; and

R⁸ particularly is trityl, 2-chlorotrityl, 4-methyltrityl, but more particularly trityl.

The resin bound peptide precursor of the formula II can be prepared using methods known to the skilled in the art of solid phase peptide synthesis, usually by a repeated Fmoc cleavage and a repeated coupling of the desired Fmoc protected amino acids.

As a rule commercially available amide resins suitable for solid phase peptide synthesis, particularly for Fmoc solid phase peptide synthesis can be used. Useful resins are for instance described in Chan W. C. and White P. D. “Fmoc Solid Phase Peptide Synthesis”, Oxford University Press. For example the PL-Rink resin (4-[(2,4-Dimethoxyphenyl)Fmoc-aminomethyl] phenoxyacetamido methyl resin) from Agilent Technology was found to be particular suitable for the process of the present invention.

Fmoc cleavage can happen with a solution of piperidine derivatives in a suitable organic solvent. Advantageously a piperidine or 4-methyl piperidine solution in N,N-dimethylformamide or N-methylpyrrolidone can be applied.

The coupling on the resin with the Fmoc protected amino acids can take place with a coupling agent selected from benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), hydroxybenzotriazole (HOBt) and N,N′-diisopropylcarbodiimide (DIC), N,N,N′,N′-tetramethyl-O-(benzotriazol-1-yl)-N,N,N′,N′-tetramethyluronium hexafluorophosphate (HBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), tetramethylfluoroformamidinium hexafluorophosphate (TFFH), 2-hydrox-pyridine (HOPy) or 4-(4,6-dimethoxy-1,3,5-triazin-2-yl)-4-methylmorpholinium chloride (DMTMM) in the presence of an organic amine base and a suitable organic solvent.

HOBt, HOPy and DIC in the presence of pyridine as organic amine base and N,N′-dimethlyformamide as organic solvent has been found to be a preferred coupling agent.

The Fmoc-Gly-Tyr(tBu)-Ile-Gln(Trt)-Asn(Trt)-Glu(OAll)-Gly-Leu-Gly-resin of formula X

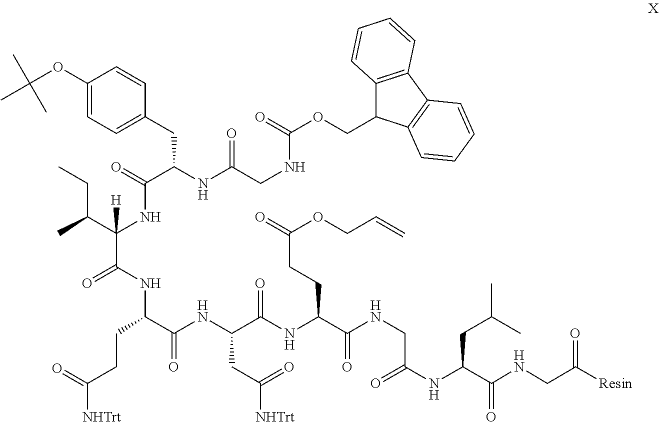

can for instance be built on a PL-Rink resin by repeated Fmoc cleavage and repeated coupling of the following Fmoc-protected amino acids in the order described: Fmoc-Gly-OH, Fmoc-Leu-OH, Fmoc-Gly-OH, Fmoc-Glu(OAll)-OH, Fmoc-Asn(Trt)-OH, Fmoc-Gln(Trt)-OH, Fmoc-Ile-OH, Fmoc-Tyr(tBu)-OH and Fmoc-Gly-OH.

As outline above, the process of the present invention can follow method a) wherein R⁶ is allyl or 4-{N-[1-(4,4-dimethyl-2,6-dioxocyclohexylidene)-3-methylbutyl]amino}benzyl. In this case the method is characterized by the following steps:
a¹) the allyl or 4-{N-[1-(4,4-dimethyl-2,6-dioxocyclohexylidene)-3-methylbutyl]amino}benzyl group R⁶ is cleaved, in a subsequent step
a²) the Fmoc group R⁵ is cleaved, thereafter
a³) ring cyclization is effected on the resin, in a further step
a⁴) global deprotection and cleavage from the resin is effected, and optionally
a⁵) the oxytocin analogue of formula I so obtained is purified and isolated.

The allyl or 4-{N-[1-(4,4-dimethyl-2,6-dioxocyclohexylidene)-3-methylbutyl]amino}benzyl group cleavage in step a¹) is usually performed in presence of a palladium or a rhodium compound or of hydrazine. Suitable palladium or rhodium compounds can be selected from tetrakis(triphenylphosphine) palladium, palladium acetate/triphenylphosphine, palladium acetate/triethylphosphite, bis(triphenylphosphine)palladium dichloride or tris(triphenylphosphine)rhodium chloride. Preferably palladium compounds, even more preferably tetrakis(triphenylphosphine) palladium are used.

In addition a scavenger such as phenylsilane, pyrrolidine, morpholine or N-methyl-N-trimethyl silyl-trifluoroacetamide, particularly phenylsilane is usually present.

The reaction as a rule can happen at room temperature in a suitable organic solvent such as methylene chloride, acetonitrile or tetrahydrofuran.

The Fmoc cleavage in step a²) can be performed as outlined above with piperidine or 4-methyl-piperidine in a suitable organic solvent.

The ring cyclization in step a³) is effected on the resin, expediently using a cyclization agent selected from benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), N,N,N′,N′-tetramethyl-O-(1H-benzotriazol-1-yl)uranium hexafluorophosphate (HBTU), 1-[bis(dimethylamino)methylene]-1H-1,2,3-triazolo[4,5-b]pyridinium 3-oxid hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), 2-hydroxy-pyridine (HOPy) or 4-(4,6-dimethoxy-1,3,5-triazin-2-yl)-4-methylmorpholinium chloride (DMTMM) in the presence of an organic amine base.

Suitable organic amine bases can be selected from pyridine, imidazole, N,N-diisopropylethyl amine, triethylamine, N-methylmorpholine, N,N-dimethyl-4-aminopyridine, 1,8-Diazabicyclo[5.4.0]undec-7-ene or 1,4-diazabicyclo[2.2.2]octane.

In a preferred embodiment the cyclization step a³) can be performed with PyBOP or PyAOP in the presence of N,N-diisopropylethyl amine, imidazole or N-methylmorpholine as organic amine bases at temperatures between 0° C. to 25° C.

Global deprotection and cleavage from the resin in step a⁴) can be effected in the presence of trifluoroacetic acid/water and a suitable scavenger such as thioanisole, anisole, phenol, triisopropylsilane, triethylsilane, ethanedithiol or dithiothreitol usually at temperatures between of 0° C. to 25° C. Triisopropylsilane has been found to be a preferred scavenger.

In step a⁵) the crude oxytocin analogue can be isolated by filtering off the resin, by removing the solvent from the filtrate and further by taking the residue up in a suitable organic solvent such as in methyl t-butyl ether, 2-methyltetrahydrofuran or in mixtures thereof and by final filtration and drying.

The crude oxytocin analogue can be further purified by preparative HPLC in solution with a suitable organic solvent such as with aqueous acetonitrile and suitable additives such as trifluoroacetic acid, acetic acid or ammonium acetate.

The fractions obtained can then be lyophilized to obtain pure oxytocin analogue of formula I.
Alternatively the process of the present invention can follow method b) wherein R⁶ is t-butyl, 1-adamantyl or phenylisopropyl. In this case the method is characterized by the following steps:
   b¹) the Fmoc group R⁵ is cleaved, thereafter
   b²) global deprotection and cleavage from the resin is effected, in a further step
   b³) ring cyclization is effected in solution, then optionally
   b⁴) the oxytocin analogue of formula I so obtained is isolated and purified.

The Fmoc cleavage in step b¹) can take place as described for step a²) above.

Global deprotection and cleavage from the resin in step b²) can be performed as described above in step a⁴). The preferred embodiments described for step a⁴ likewise apply for step b²).

The ring cyclization in step b³) is effected in solution but can happen with the cyclization agents and the organic amine bases listed for step a³) above. The preferred embodiments described for step a³ likewise apply for step b³).

Isolation and purification in step b⁴) can take place in the same manner as described in step a⁵). The preferred embodiments described for step a⁵ likewise apply for step b⁴).

In a particular embodiment of the present invention process alternative b) is favored over process alternative a).

### EXAMPLES

Abbreviations:

SPPS=Solid-phase peptide synthesis, PL-Rink resin=4-[(2,4-Dimethoxyphenyl)Fmoc-aminomethyl]phenoxyacetamido methyl resin from Agilent Technology (PL1467-4749: 0.32 mmol/g 75-150-10⁻⁶m; PL1467-4799: 0.55 mmol/g 75-150-10⁻⁶m; PL1467-4689: 0.96 mmol/g 150-300-10⁻⁶m), Fmoc=9-Fluorenylmethoxycarbonyl, Gly=Glycine, Leu=Leucine, Glu(OAll)=Allyl-protected glutamic acid, Glu(tBu)=tert Butyl-protected glutamic acid, Asn(Trt)=Trityl-protected asparagine, Gln(Trt)=Trityl-protected glutamine, Ile=Isoleucine, Tyr(tBu)=tert Butyl-protected tyrosine, Sar=N-methylglycine, Pro=Proline, Nle=Norleucine, DMF=N,N-Dimethylformamide, HOBt=1-Hydroxybenzotriazole, HOPy=2-hyxroxy-pyridine, DIC=N,N′-Diisopropylcarbodiimide, NEP=N-Ethylpyrrolidone, PyBOP=(Benzotriazol-1-yloxy)tripyrrolidinophosphonium hexafluorophosphate, DIPEA=Diisopropylethyl amine, MeOH=Methanol, CH₂Cl₂=Dichloromethane, MTBE=Methyl tert-butyl ether, MeTHF=2-Methyltetrahydrofuran, TFA=Trifluoroacetic acid, MeCN=Acetonitrile, PyAOP=(7-Azabenzotriazol-1-yloxy)tripyrrolidinophosphonium hexafluorophosphate, HBTU=N,N,N′,N′-Tetramethyl-O-(1H-benzotriazol-1-yl)uranium hexafluorophosphate, HATU=1-[Bis(dimethylamino) methylene]-1H-1,2,3-triazolo[4,5-b]pyridinium 3-oxid hexafluorophosphate, HCTU=O-(6-Chlorobenzotriazol-1-yl)-N,N,N′,N′-tetramethyluronium hexafluorophosphate, COMU=(1-Cyano-2-ethoxy-2-oxoethylidenaminooxy)dimethylamino-morpholino-carbenium hexafluorophosphate, DMTMM=4-(4,6-Dimethoxy-1,3,5-triazin-2-yl)-4-methylmorpholinium chloride, NMP=1-Methyl-2-pyrrolidinone, DMSO=Dimethyl sulfoxide, DMI=1,3-Dimethyl-2-imidazolidinone, DMPU=1,3-Dimethyl-3,4,5,6-tetrahydro-2(1H)-pyrimidinone, NMM=N-Methylmorpholine, DMAP=N,N-Dimethyl-4-aminopyridine, DIPEA=N,N-Diisopropylethylamine, DBU=1,8-Diazabicyclo[5.4.0]undec-7-ene, DABCO=1,4-Diazabicyclo[2.2.2]octane.

### Comparison Example

A comparative experiment was run for the preparation of

c[Gly-Tyr-Ile-Gln-Asn-Glu]-Gly-Leu-Gly-NH₂ (1)

in analogy to the synthesis description of the WO2014/095773 (Solid phase cyclization) and as outlined in scheme 1 below:

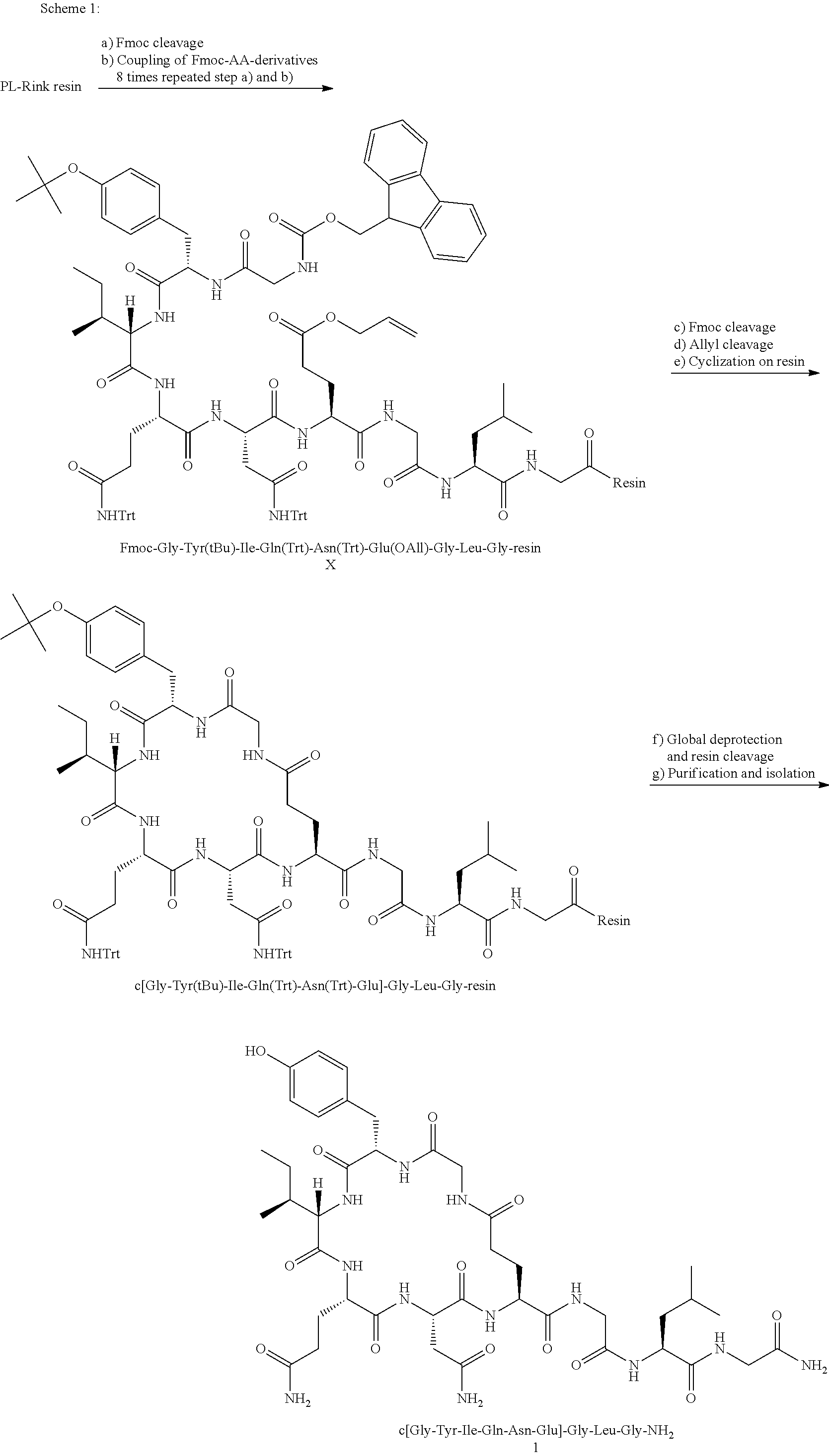

Synthesis performance has been measured based on the yield and the ratio of product (1) to the dimer-by product of the formula shown in scheme 2 below:

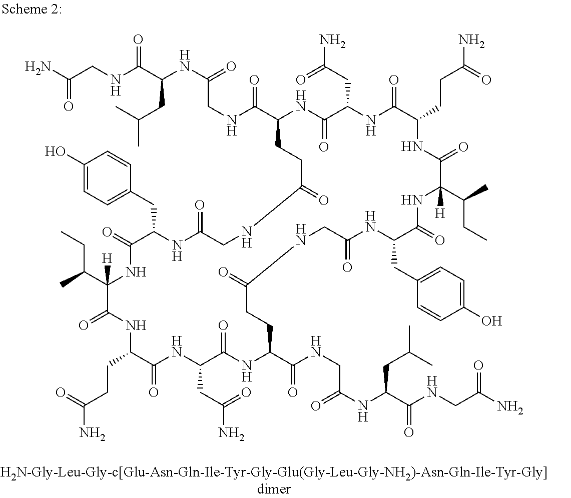 [SMILES: CC[C@H](C)[C@@H]1NC(=O)[C@H](Cc2ccc(O)cc2)NC(=O)CNC(=O)CC[C@@H](C(=O)NCC(=O)N[C@@H](CC(C)C)C(=O)NCC(N)=O)NC(=O)[C@H](CC(N)=O)NC(=O)[C@H](CCC(N)=O)NC(=O)[C@H]([C@@H](C)CC)NC(=O)[C@H](Cc2ccc(O)cc2)NC(=O)CNC(=O)CC[C@@H](C(=O)NCC(=O)N[C@@H](CC(C)C)C(=O)NCC(N)=O)NC(=O)[C@H](CC(N)=O)NC(=O)[C@H](CCC(N)=O)NC1=O]

a) Fmoc-Cleavage:

A SPPS reactor (100 mL; peptide synthesizer CS136XT ex CSBio) was charged with PL-Rink resin (load. 0.55 mmol/g, 5.00 g, 2.75 mmol) and 20% piperidine in DMF (50.0 mL). The mixture was then stirred at 25° C. for 10 min. After draining the solvent, another portion of 20% piperidine in DMF (50.0 mL) was added and the mixture was stirred at 25° C. for 30 min. After draining the solvent, the resultant resin was washed with DMF (8×50.0 mL) to yield deFmoc-PL-Rink resin.

b) Coupling with Fmoc-AA-Derivatives:

To deFmoc-PL-Rink resin, a solution of Fmoc-Gly-OH in 0.35M HOBt/DMF (32.0 mL, 11.2 mmol), 0.92M DIC in DMF (16.0 mL, 14.7 mmol) and 10% pyridine in DMF (16.0 mL, 19.8 mmol) were added and stirred at 25° C. for 3 h. After draining the solvent, the resultant resin was washed with DMF (4×50.0 mL) to yield Fmoc-Gly-resin.

Fmoc-Cleavage and Fmoc-AA-derivative coupling steps were repeated 8 times employing instead of Fmoc-Gly-OH, the following Fmoc-amino acid-derivatives: Fmoc-Leu-OH, Fmoc-Gly-OH, Fmoc-Glu(OAll)-OH, Fmoc-Asn(Trt)-OH, Fmoc-Gln(Trt)-OH, Fmoc-Ile-OH, Fmoc-Tyr(tBu)-OH, Fmoc-Gly-OH to yield Fmoc-Gly-Tyr(tBu)-Ile-Gln(Trt)-Asn(Trt)-Glu(OAll)-Gly-Leu-Gly-resin. A sample was cleaved from the resin (vide below) to confirm the correct mass. MS (m/z): 1211.8 (M+H)⁺

c) Fmoc-Cleavage:

Fmoc-Cleavage of the terminal Gly was conducted as described above to yield H-Gly-Tyr(tBu)-Ile-Gln(Trt)-Asn(Trt)-Glu(OAll)-Gly-Leu-Gly-resin. A sample was cleaved from the resin (vehicle below) to confirm the correct mass. MS (m/z): 989.8 (M+H)⁺

d) Allyl-Cleavage:

To H-Gly-Tyr(tBu)-Ile-Gln(Trt)-Asn(Trt)-Glu(OAll)-Gly-Leu-Gly-resin, a solution of tetrakis triphenylphosphine palladium (159 mg, 0.138 mmol) and phenylsilane (3.40 mL, 27.6 mmol) in CH₂Cl₂ (50.0 mL) was added and stirred at 25° C. for 30 min. After draining the solvent, this step was repeated once more and washed with DMF (2×50.0 mL). A solution of sodium dithiocarbamate (250 mg) and DIPEA (0.250 mL) in DMF (50.0 mL) was added and the mixture was stirred at 25° C. for 15 min. After draining the solvent, this step was repeated once more. After draining the solvent, the resultant resin was washed with DMF (4×50.0 mL) to yield H-Gly-Tyr(tBu)-Ile-Gln(Trt)-Asn(Trt)-Glu-Gly-Leu-Gly-resin. A sample was cleaved from the resin (vehicle below) to confirm the correct mass. MS (m/z): 949.7 (M+H)⁺

e) Cyclization on Resin:

A solution of PyBOP (2.36 g, 4.54 mmol) and DIPEA (2.40 mL, 13.8 mmol) in NEP (60.0 mL) was added to H-Gly-Tyr(tBu)-Ile-Gln(Trt)-Asn(Trt)-Glu-Gly-Leu-Gly-resin and the mixture was stirred at 25° C. for 4 h. After draining the solvent, the resultant resin was washed with DMF (4×50.0 mL), CH₂Cl₂ (3×50.0 mL) and MeOH (3×50.0 mL). The resin was dried at 10 mbar at 25° C. for 1 day to afford c[Gly-Tyr(tBu)-Ile-Gln(Trt)-Asn(Trt)-Glu]-Gly-Leu-Gly-resin (8.60 g).

f) Global Deprotection and Resin Cleavage:

To a precooled (10-15° C.) solution of triisopropylsilane (2.80 mL) in TFA (40.0 mL) and water (10.0 mL), c[Gly-Tyr(tBu)-Ile-Gln(Trt)-Asn(Trt)-Glu]-Gly-Leu-Gly-resin (8.60 g) was added and stirred at 25° C. for 3 h. The resin was filtered off and the filtrate was concentrated in vacuo. The residue was added to MTBE (100 mL) and the mixture was stirred at 25° C. for 15 h. The mixture was filtered and the cake was washed with MTBE (50.0 mL) followed by drying to afford crude c[Gly-Tyr-Ile-Gln-Asn-Glu]-Gly-Leu-Gly-NH₂ 1 (2.01 g, assay 11.3 wt %, total 9% yield) as a white solid with 15.9% purity (HPLC area-%, HPLC method cf. Example 1). The ratio of 1/dimer was 8.5.

TABLE 1

| | | Total | Purity of crude | |
|---|---|---|---|---|
| | Cyclization | yield | cyclic peptide | Ratio of |
| Example | method | (%) | (HPLC area-%) | 1/dimer |
| Comparison | Solid phase | 9 | 15.9 | 8.5 |
| 1 (invention) | Solid phase | 38 | 62.7 | 21.9 |
| 2 (invention) | Liquid phase | 31 | 56.6 | 15.1 |

### Example 1

### Solid Phase Cyclization

c[Gly-Tyr-Ile-Gln-Asn-Glu]-Gly-Leu-Gly-NH₂ (1)

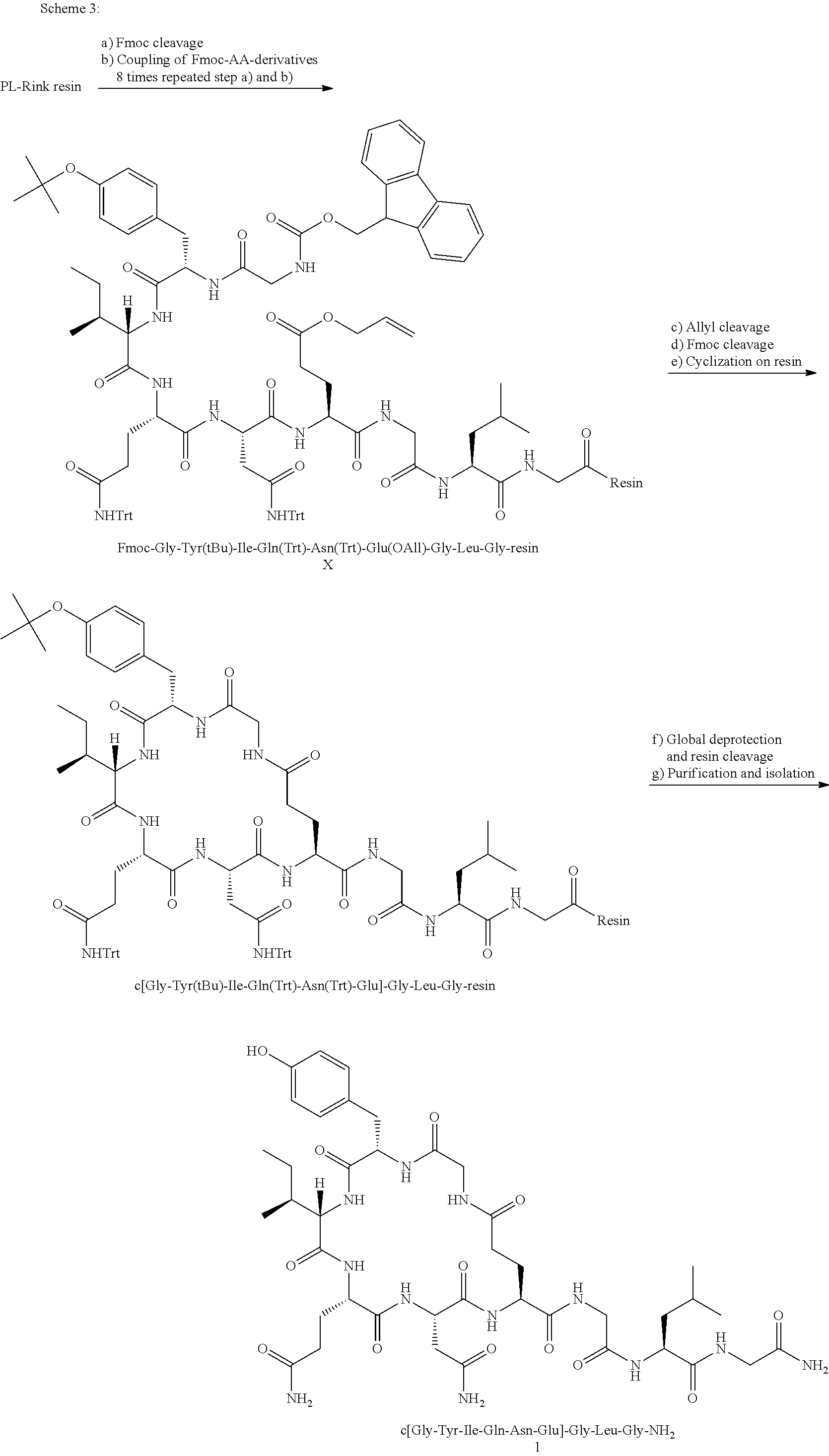

a) Fmoc-Cleavage:

A SPPS reactor (100 mL; peptide synthesizer CS136XT ex CSBio) was charged with PL-Rink resin (load. 0.55 mmol/g, 5.00 g, 2.75 mmol) and 20% piperidine in DMF (50.0 mL). The mixture was then stirred at 25° C. for 10 min. After draining the solvent, another portion of 20% piperidine in DMF (50.0 mL) was added and the mixture was stirred at 25° C. for 30 min. After draining the solvent, the resultant resin was washed with DMF (8×50.0 mL) to yield deFmoc-PL-Rink resin.

b) Coupling with Fmoc-AA-Derivatives:

To deFmoc-PL-Rink resin, a solution of Fmoc-Gly-OH in 0.35M HOBt/DMF (32.0 mL, 11.2 mmol), 0.92M DIC in DMF (16.0 mL, 14.7 mmol) and 10% pyridine in DMF (16.0 mL, 19.8 mmol) were added and stirred at 25° C. for 3 h. After draining the solvent, the resultant resin was washed with DMF (4×50.0 mL) to yield Fmoc-Gly-resin.

Fmoc-Cleavage and Fmoc-AA-derivative coupling steps were repeated 8 times employing instead of Fmoc-Gly-OH, the following Fmoc-amino acid-derivatives: Fmoc-Leu-OH, Fmoc-Gly-OH, Fmoc-Glu(OAll)-OH, Fmoc-Asn(Trt)-OH, Fmoc-Gln(Trt)-OH, Fmoc-Ile-OH, Fmoc-Tyr(tBu)-OH, Fmoc-Gly-OH to yield X (Fmoc-Gly-Tyr(tBu)-Ile-Gln(Trt)-Asn(Trt)-Glu(OAll)-Gly-Leu-Gly-resin). A sample was cleaved from the resin (vide below) to confirm the correct mass. MS (m/z): 1211.8 (M+H)⁺

Allyl-Cleavage:

To X (Fmoc-Gly-Tyr(tBu)-Ile-Gln(Trt)-Asn(Trt)-Glu(OAll)-Gly-Leu-Gly-resin), a solution of tetrakis triphenylphosphine palladium (159 mg, 0.138 mmol) and phenylsilane (3.40 mL, 27.6 mmol) in CH₂Cl₂ (50.0 mL) was added and stirred at 25° C. for 30 min. After draining the solvent, this step was repeated once more and washed with DMF (2×50.0 mL). A solution of sodium dithiocarbamate (250 mg) and DIPEA (0.250 mL) in DMF (50.0 mL) was added and the mixture was stirred at 25° C. for 15 min. After draining the solvent, this step was repeated once more. After draining the solvent, the resultant resin was washed with DMF (4×50.0 mL) to yield Fmoc-Gly-Tyr(tBu)-Ile-Gln(Trt)-Asn(Trt)-Glu-Gly-Leu-Gly-resin. A sample was cleaved from the resin (vehicle below) to confirm the correct mass. MS (m/z): 1171.8 (M+H)⁺

d) Fmoc-Cleavage:

Fmoc-Cleavage of the terminal Gly was conducted as described above to yield H-Gly-Tyr(tBu)-Ile-Gln(Trt)-Asn(Trt)-Glu-Gly-Leu-Gly-resin. A sample was cleaved from the resin (vehicle below) to confirm the correct mass. MS (m/z): 949.7 (M+H)⁺

e) Cyclization on Resin:

A solution of PyBOP (2.36 g, 4.54 mmol) and DIPEA (2.40 mL, 13.8 mmol) in NEP (60.0 mL) was added to (H-Gly-Tyr(tBu)-Ile-Gln(Trt)-Asn(Trt)-Glu-Gly-Leu-Gly-resin and the mixture was stirred at 25° C. for 4 h. After draining the solvent, the resultant resin was washed with DMF (4×50.0 mL), CH₂Cl₂ (3×50.0 mL) and MeOH (3×50.0 mL). The resin was dried at 10 mbar at 25° C. for 1 day to afford c[Gly-Tyr(tBu)-Ile-Gln(Trt)-Asn(Trt)-Glu]-Gly-Leu-Gly-resin (9.17 g).

f) Global Deprotection and Resin Cleavage:

To a precooled (10-15° C.) solution of triisopropylsilane (2.50 mL) in TFA (40.0 mL) and water (10.0 mL), c[Gly-Tyr(tBu)-Ile-Gln(Trt)-Asn(Trt)-Glu]-Gly-Leu-Gly-resin (9.17 g) was added and stirred at 25° C. for 3 h. The resin was filtered off and the filtrate was concentrated in vacuo. The residue was added to MTBE (100 mL) and the mixture was stirred at 25° C. for 15 h. The mixture was filtered and the cake was washed with MTBE (50.0 mL) followed by drying to afford crude c[Gly-Tyr-Ile-Gln-Asn-Glu]-Gly-Leu-Gly-NH₂ 1 (2.39 g, assay 40.9 wt %, total 38% yield) as a white solid with 62.7% purity (HPLC area-%, HPLC method: Aquity UPLC BEH130 C18 column, 150×2.1 mm; mobile phase, A: 0.05% TFA in water, B: 0.05% TFA in MeCN; flow: 0.13 mL/min for 40 min, 0.25 mL/min for 15 min; isocratic 90/10 (A/B) for 3 min, gradient from 90/10 (A/B) to 62/38 (A/B) within 37 min, gradient from 62/38 (A/B) to 10/90 (A/B) within 5 min, isocratic 10/90 (A/B) for 10 min. Temp: 60° C., UV:214 nm). The ratio of 1/dimer was 21.9.

Retention time: 23.2 min (c[Gly-Tyr-Ile-Gln-Asn-Glu]-Gly-Leu-Gly-NH₂), 18.8 min (H-Gly-Tyr-Ile-Gln-Asn-Glu-Gly-Leu-Gly-NH₂), 26.1 min (dimer)

g) Purification and Isolation:

Crude c[Gly-Tyr-Ile-Gln-Asn-Glu]-Gly-Leu-Gly-NH₂ was dissolved in water-MeCN (10-1) and filtered. The filtrate was diluted with the same volume of water. The solution was purified by preparative HPLC on a Kromasil-C18-100 column (250×80 mm, 10 um particle size, A: 0.1% TFA-water, B: MeCN; flow: 300 mL/min; isocratic 95/5 (A/B) for 2 min, gradient from 95/5 (A/B) to 80/20 (A/B) within 1 min, gradient from 80/20 (A/B) to 77/23 (A/B) within 17 min, gradient from 77/23 (A/B) to 10/90 (A/B) within 1 min, isocratic 10/90 (A/B) for 7 min, gradient from 10/90 (A/B) to 95/5 (A/B) within 1 min, isocratic 95/5 (A/B) for 6 min. The fractions were collected and lyophilized to yield pure c[Gly-Tyr-Ile-Gln-Asn-Glu]-Gly-Leu-Gly-NH₂ 1 (0.708 g) as a white powder with 99.2% purity (HPLC area-%, HPLC method cf. Example 1). No dimer was observed in pure 1. MS (m/z): 931.0 (M+H)⁺

### Example 2

### Solution Phase Cyclization

c[Gly-Tyr-Ile-Gln-Asn-Glu]-Gly-Leu-Gly-NH₂ (1)

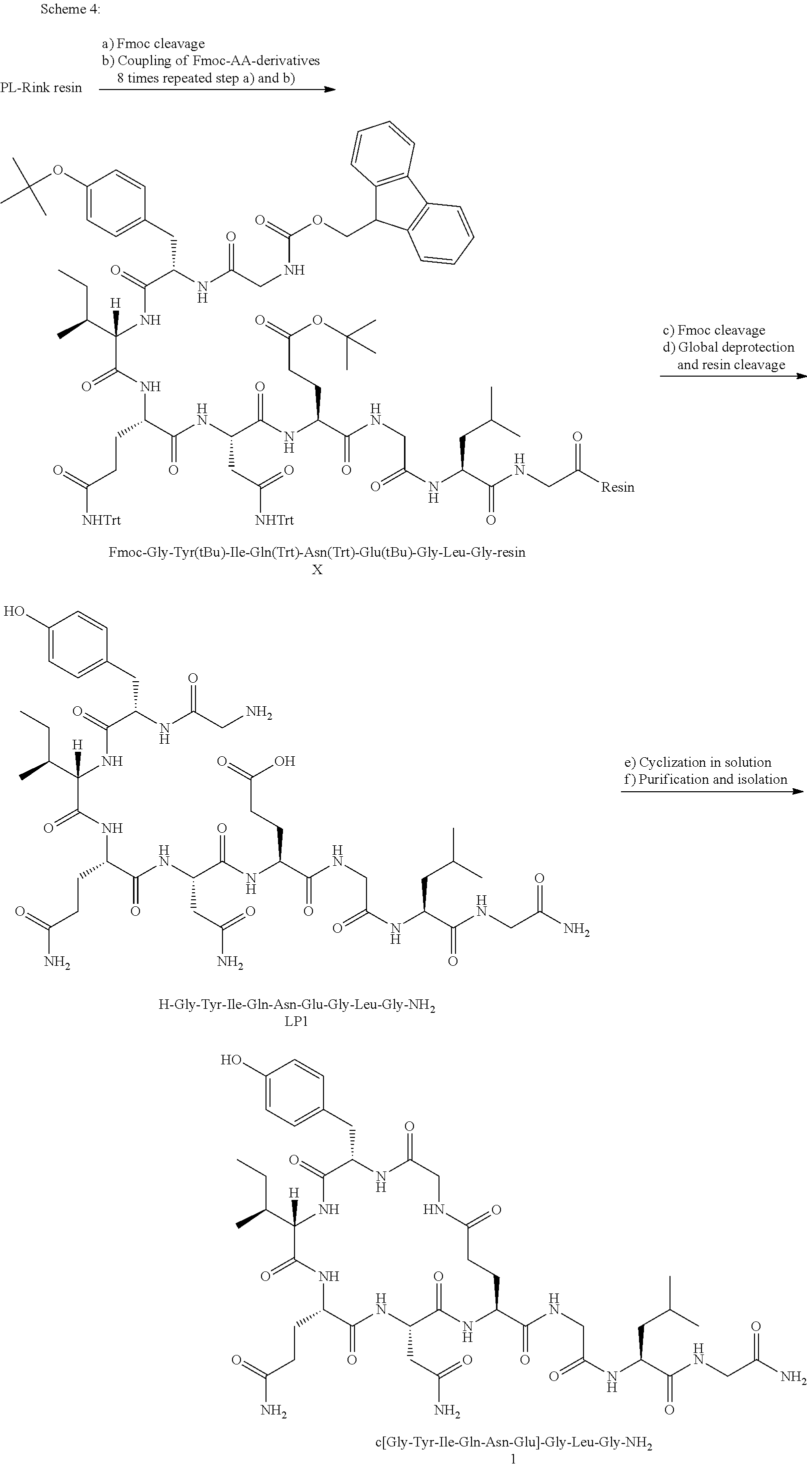

a) Fmoc-Cleavage:

A SPPS reactor (100 mL) was charged with PL-Rink resin (load. 0.55 mmol/g, 5.00 g, 2.75 mmol) and 20% piperidine in DMF (50 mL). The mixture was then stirred at 25° C. for 10 min. After draining the solvent, another portion of 20% piperidine in DMF (50.0 mL) was added and the mixture was stirred at 25° C. for 30 min. After draining the solvent, the resultant resin was washed with DMF (8×50.0 mL) to yield deFmoc-PL-Rink-resin.

b) Coupling of Fmoc-AA-Derivatives:

To deFmoc-PL-Rink-resin, a solution of Fmoc-Gly-OH in 0.35M HOBt/DMF (32.0 mL, 11.2 mmol), 0.92M DIC in DMF (16.0 mL, 14.7 mmol) and 10% pyridine in DMF (16.0 mL, 19.8 mmol) were added and stirred at 25° C. for 3 h. After draining the solvent, the resultant resin was washed with DMF (4×50.0 mL) to yield Fmoc-Gly-resin.

Fmoc-Cleavage and Fmoc-AA-derivative coupling steps were repeated 8 times employing instead of Fmoc-Gly-OH, the following Fmoc-amino acid-derivatives: Fmoc-Leu-OH, Fmoc-Gly-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH to yield X (Fmoc-Gly-Tyr(tBu)-Ile-Gln(Trt)-Asn(Trt)-Glu(tBu)-Pro-Leu-Gly-resin). A sample was cleaved from the resin (vide below) to confirm the correct mass. MS (m/z): 1171.8 (M+H)⁺

c) Fmoc-Cleavage:

Fmoc-Cleavage of the terminal Gly was conducted as described above. After draining the solvent, the resultant resin was washed with DMF (8×50.0 mL), CH₂Cl₂ (3×50.0 mL) and MeOH (3×50.0 mL). The resin was dried at 10 mbar at 25° C. for 1 day to afford to yield H-Gly-Tyr(tBu)-Ile-Gln(Trt)-Asn(Trt)-Glu(tBu)-Gly-Leu-Gly-resin (10.8 g).

d) Global Deprotection and Resin Cleavage:

To a precooled (10-15° C.) solution of triisopropylsilane (2.50 mL) in TFA (40.0 mL) and water (10.0 mL), H-Gly-Tyr(tBu)-Ile-Gln(Trt)-Asn(Trt)-Glu(tBu)-Gly-Leu-Gly-resin (10.8 g) was added and stirred at 25° C. for 3 h. The resin was filtered off and the filtrate was concentrated in vacuo. The residue was added to MeTHF (100 mL) and the mixture was stirred at 25° C. for 15 h. The mixture was filtered and the cake was washed with MeTHF (50.0 mL) followed by drying to afford LP1 (H-Gly-Tyr-Ile-Gln-Asn-Glu-Gly-Leu-Gly-NH₂) (3.60 g) as a white solid with 67.4% purity (HPLC area-%, HPLC method cf. example 1). MS (m/z): 949.7 (M+H)⁺

e) Cyclization in Solution:

To a mixture of LP1 (H-Gly-Tyr-Ile-Gln-Asn-Glu-Gly-Leu-Gly-NH₂) (3.50 g) in NEP (60.0 mL) and DIPEA (3.13 mL, 18.4 mmol) was added PyBOP (1.92 g, 3.69 mmol) and stirred at 25° C. for 1 h. To complete conversion, another portion of PyBOP (0.960 g, 1.84 mmol) was added and stirred at the same temperature for 1 h. The resultant mixture was added to a solution of MTBE/MeTHF solution (400 mL/100 mL) and stirred at 25° C. for 15 h. The mixture was filtered and the cake was washed with MTBE (50.0 mL) followed by drying to afford crude c[Gly-Tyr-Leu-Gln-Asn-Glu]-Gly-Leu-Gly-NH₂ 1(4.30 g, assay 18.0 wt %, total 31% yield) as a white solid with 56.6% purity (HPLC area-%, HPLC method cf. example 1). The ratio of 1/dimer was 15.1.

f) Purification and Isolation:

Crude c[Gly-Tyr-Leu-Gln-Asn-Glu]-Gly-Leu-Gly-NH₂ was dissolved in water-MeCN (10-1) and filtered off undissolved material. The filtrate was diluted with the same volume of water. The solution was purified by preparative HPLC on a Kromasil-C18-100 column (250×80 mm, 10 um particle size, A: 0.1% TFA-water, B: MeCN; flow: 300 mL/min; isocratic 95/5 (A/B) for 2 min, gradient from 95/5 (A/B) to 80/20 (A/B) within 1 min, gradient from 80/20 (A/B) to 77/23 (A/B) within 17 min, gradient from 77/23 (A/B) to 10/90 (A/B) within 1 min, isocratic 10/90 (A/B) for 7 min, gradient from 10/90 (A/B) to 95/5 (A/B) within 1 min, isocratic 95/5 (A/B) for 6 min. The fractions were collected and lyophilized to yield pure c[Gly-Tyr-Leu-Gln-Asn-Glu]-Gly-Leu-Gly-NH₂ 1 (444 mg) as a white powder with 99.7% purity (HPLC area-%, HPLC method cf. Example 1). No dimer was present in pure 1. MS (m/z): 931.0

### Example 3 a-g

### Optimization of Coupling Reagents

c[Gly-Tyr-Ile-Gln-Asn-Glu]-Gly-Leu-Gly-NH₂ (1)

In an analogous manner to Example 2, the cyclizations were performed employing the coupling reagents as listed in Table 2.

TABLE 2

| | | Yield in | Purity in | |
|---|---|---|---|---|
| | | reaction | reaction | |
| | Coupling | mixture | mixture | Ratio of |
| Example | reagent | (%) | (HPLC area-%) | 1/dimer |
| 3a | PyBOP | 63 | 54.9 | 13.4 |
| 3b | PyAOP | 63 | 52.7 | 23.2 |
| 3c | HBTU | 45 | 51.0 | 12.4 |
| 3d | HATU | 51 | 47.1 | 10.6 |
| 3e | HCTU | 55 | 10.6 | 13.8 |
| 3f | COMU | 26 | 11.5 | 28.0 |
| 3g | DMTMM | 17 | 18.5 | 34.9 |

### Example 4 a-g

### Optimization of Solvents

c[Gly-Tyr-Ile-Gln-Asn-Glu]-Gly-Leu-Gly-NH₂ (1)

In an analogous manner to Example 2, the cyclizations were performed employing the solvents as listed in Table 3.

TABLE 3

| | | Yield in | Purity in | |
|---|---|---|---|---|
| | | reaction | reaction | |
| | Solvent | mixture | mixture | Ratio of |
| Example | (Concentration) | (%) | (HPLC area-%) | 1/dimer |
| 4a | NEP (5 mM) | 63 | 60.3 | 30.4 |
| 4b | NEP (80 mM) | 63 | 54.9 | 13.4 |
| 4c | NMP (80 mM) | 44 | 46.9 | 13.3 |
| 4d | DMSO (80 mM) | 51 | 52.8 | 13.8 |
| 4e | DMF (80 mM) | 35 | 44.8 | 8.9 |
| 4f | DMI (80 mM) | 38 | 44.5 | 11.3 |
| 4g | DMPU (80 mM) | 35 | 45.3 | 17.7 |

### Example 5 a-g

### Optimization of Bases

c[Gly-Tyr-Ile-Gln-Asn-Glu]-Gly-Leu-Gly-NH₂ (1)

In an analogous manner to Example 2, the cyclizations were performed employing the bases as listed in Table 4.

TABLE 4

| | | Yield in | Purity in | |
|---|---|---|---|---|
| | | reaction | reaction | |
| | | mixture | mixture | Ratio of |
| Example | Base | (%) | (HPLC area-%) | 1/dimer |
| 5a | Imidazole | 63 | 55.7 | 10.6 |
| 5b | NMM | 68 | 57.7 | 22.0 |
| 5c | DABCO | 47 | 46.4 | 15.4 |
| 5d | DMAP | 43 | 47.8 | 13.6 |
| 5e | DIPEA | 63 | 54.9 | 13.4 |
| 5f | DBU | 25 | 27.0 | 19.4 |
| 5g | NMM, 0° C. | 66 | 54.5 | 20.0 |

### Example 6 a-d

### Comparison of the Resin Loading/Amino Acid Equivalent

c[Gly-Tyr-Ile-Gln-Asn-Glu]-Pro-Leu-Gly-NH₂ (2)

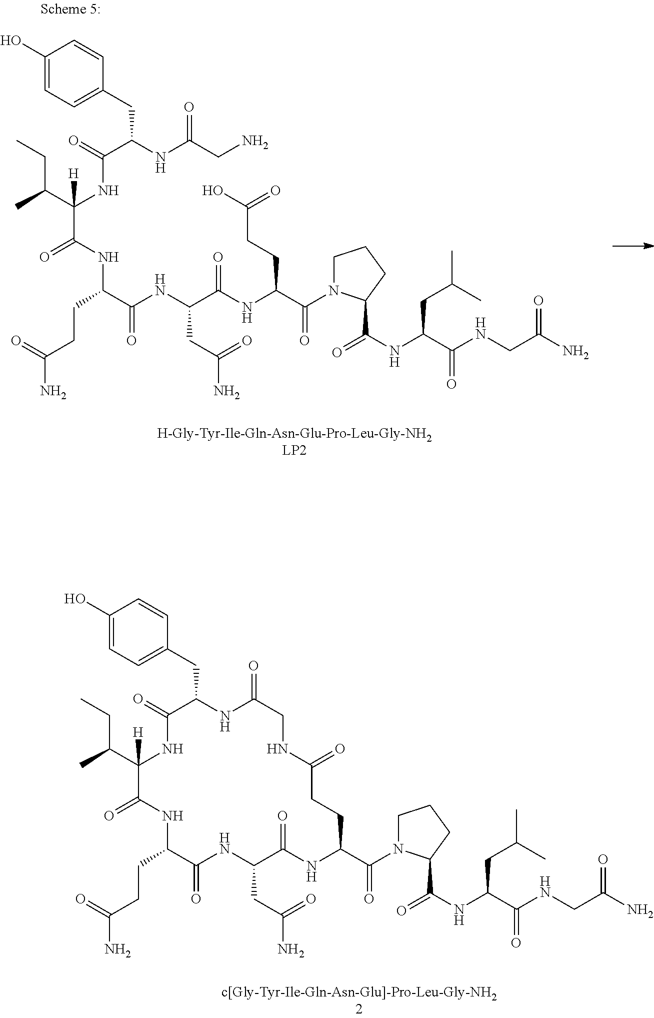 [SMILES: CC[C@H](C)[C@H](NC(=O)[C@H](Cc1ccc(O)cc1)NC(=O)CN)C(=O)N[C@@H](CCC(N)=O)C(=O)N[C@@H](CC(N)=O)C(=O)N[C@@H](CCC(=O)O)C(=O)N1CCC[C@H]1C(=O)N[C@@H](CC(C)C)C(=O)NCC(N)=O ; CC[C@H](C)[C@@H]1NC(=O)[C@H](Cc2ccc(O)cc2)NC(=O)CNC(=O)CC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

In an analogous manner to Example 2, pure cyclic peptide 2 was synthesized employing the Fmoc-AA-acids: Fmoc-Gly-OH, Fmoc-Leu-OH, Fmoc-Pro-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH

Scale of synthesis: 9.60 mmol (load: see example 6a-d; resin 30.0 g)

Yield: 40% (after purification)

Purity: 98.2% (HPLC area-%, HPLC method cf. example 1)

Retention time: 29.8 min (HPLC method cf. Example 1)

MS (m/z): 971.5 (M+H)⁺

Purity and yield of the linear peptide intermediate LP2 (H-Gly-Tyr-Ile-Gln-Asn-Glu-Pro-Leu-Gly-NH₂) was determined employing the resin loadings/amino acid equivalents as listed in Table 5.

TABLE 5

| | Loading of | Amino | Purity of | Yield of |
|---|---|---|---|---|
| | resin | acid | crude LP2 | crude LP2 |
| Example | (mmol/g) | (eq.) | (HPLC area-%) | (%) |
| 6a | 0.32 | 4 | 79.0 | 90 |
| 6b | 0.55 | 4 | 83.3 | 116 |
| 6c | 0.55 | 2 | 78.5 | 108 |
| 6d | 0.96 | 4 | 82.7 | 96 |

### Example 7

c[Gly-Tyr-Ile-Gln-Asn-Glu]-Pro-Leu-Gly-NH₂ (2)

Example 7 was performed in an analogous manner to Example 2, with the exception that the cyclizations were performed employing N-methylmorpholine as base.

a) Fmoc-Cleavage:

A SPPS reactor (250 mL; peptide synthesizer CS536XT ex CSBio) was charged with PL-Rink resin (load. 0.55 mmol/g, 10.0 g, 5.50 mmol) and 20% piperidine in DMF (100 mL). The mixture was then stirred at 25° C. for 10 min. After draining the solvent, another portion of 20% piperidine in DMF (100 mL) was added and the mixture was stirred at 25° C. for 30 min. After draining the solvent, the resultant resin was washed with DMF (8×100 mL) to yield deFmoc-PL-Rink-resin.

b) Coupling of Fmoc-AA-Derivatives:

To deFmoc-PL-Rink-resin, a solution of Fmoc-Gly-OH in 0.35M HOBt/DMF (64.0 mL, 22.4 mmol), 0.92M DIC in DMF (32.0 mL, 29.4 mmol) and 10% pyridine in DMF (32.0 mL, 39.6 mmol) were added and stirred at 25° C. for 3 h. After draining the solvent, the resultant resin was washed with DMF (4×100 mL) to yield Fmoc-Gly-resin.

Fmoc-Cleavage and Fmoc-AA-derivative coupling steps were repeated 8 times employing instead of Fmoc-Gly-OH, the following Fmoc-amino acid-derivatives: Fmoc-Leu-OH, Fmoc-Pro-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH to yield Fmoc-Gly-Tyr(tBu)-Ile-Gln(Trt)-Asn(Trt)-Glu(tBu)-Pro-Leu-Gly-resin. A sample was cleaved from the resin (vehicle below) to confirm the correct mass. MS (m/z): 1211.1 (M+H)⁺

c) Fmoc-Cleavage:

Fmoc-Cleavage of the terminal Gly was conducted as described above. After draining the solvent, the resultant resin was washed with DMF (8×100 mL), CH₂Cl₂ (3×100 mL) and MeOH (3×100 mL). The resin was dried under 10 mbar at 25° C. for 1 day to afford to yield H-Gly-Tyr(tBu)-Ile-Gln(Trt)-Asn(Trt)-Glu(tBu)-Pro-Leu-Gly-resin (18.6 g). A sample was cleaved from the resin (vehicle below) to confirm the correct mass. MS (m/z): 989.7 (M+H)⁺

d) Global Deprotection and Resin Cleavage:

To a precooled (10-15° C.) solution of triisopropylsilane (3.00 mL) in TFA (48.0 mL) and water (12.0 mL), H-Gly-Tyr(tBu)-Ile-Gln(Trt)-Asn(Trt)-Glu(tBu)-Pro-Leu-Gly-resin (6.00 g) was added and stirred at 25° C. for 3 h. The resin was filtered off and the filtrate was concentrated in vacuo. The residue was added to MeTHF (120 mL) and the mixture was stirred at 25° C. for 15 h. The mixture was filtered and the cake was washed with MeTHF (60.0 mL) followed by drying to afford H-Gly-Tyr-Ile-Gln-Asn-Glu-Pro-Leu-Gly-NH₂ LP2 (1.84 g) as a white solid with 87.3% purity (HPLC area-%, HPLC method cf. Example 1). Retention time: 23.9 min (HPLC method cf. Example 1); MS (m/z): 989.7 (M+H)⁺

e) Cyclization in Solution:

To a mixture of H-Gly-Tyr-Ile-Gln-Asn-Glu-Pro-Leu-Gly-NH₂ LP2 (300 mg) in N-ethylpyrrolidone (3.60 mL) and NMM (0.167 mL, 1.52 mmol) was added PyBOP (237 mg, 0.455 mmol) and stirred at 25° C. for 1 h. To complete conversion, another portion of PyBOP (47.4 mg, 0.0910 mmol) was added and stirred at the same temperature for 1 h. The resultant mixture was added to a solution of MTBE (24.0 mL) and MeTHF (6.00 mL), and then stirred at 25° C. for 15 h. The mixture was filtered and the cake was washed with MTBE (15.0 mL). The cake was dissolved in water/MeCN (10/1, 3.3 mL) and filtered off undissolved materials. The filtrate was lyophilized to afford crude c[Gly-Tyr-Leu-Gln-Asn-Glu]-Pro-Leu-Gly-NH₂ 2 (313 mg, assay 54.0 wt %, total 60% yield) as a white solid with 71.4% purity (HPLC area-%, HPLC method cf. Example 1).MS (m/z): 971.5 (M+H)⁺

### Example 8

c[Gly-Tyr-Ile-Gln-Asn-Glu]-Sar-Leu-Gly-NH₂ (3)

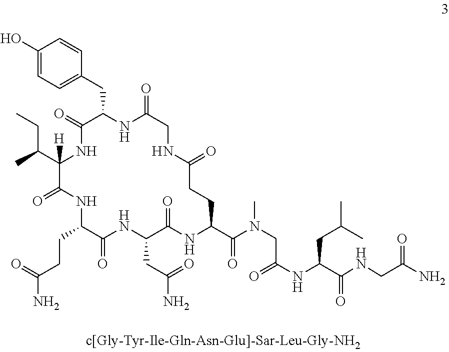 [SMILES: CC[C@H](C)[C@@H]1NC(=O)[C@H](Cc2ccc(O)cc2)NC(=O)CNC(=O)CC[C@@H](C(=O)N(C)CC(=O)N[C@@H](CC(C)C)C(=O)NCC(N)=O)NC(=O)[C@H](CC(N)=O)NC(=O)[C@H](CCC(N)=O)NC1=O]

In an analogous manner to Example 2, pure cyclic peptide 3 was synthesized employing the Fmoc-AA-acids: Fmoc-Gly-OH, Fmoc-Leu-OH, Fmoc-Sar-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH

Scale of synthesis: 9.60 mmol (load. 0.32 mmol/g, resin 30.0 g)

Yield: 41% (after purification)

Purity: 98.9% (HPLC area-%, HPLC method cf. Example 1)

Retention time: 27.6 min (HPLC method cf. Example 1)

MS (m/z): 945.5 (M+H)⁺

### Example 9

c[Gly-Tyr-Ile-Gln-Asn-Glu]-Sar-Nle-Gly-NH₂ (4)

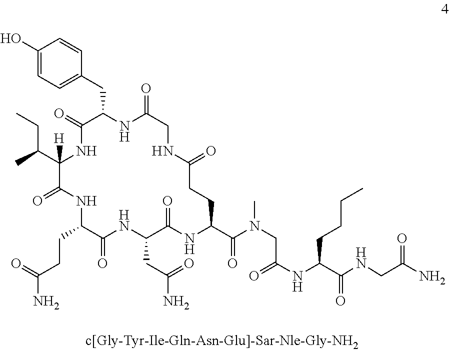 [SMILES: CCCC[C@H](NC(=O)CN(C)C(=O)[C@@H]1CCC(=O)NCC(=O)N[C@@H](Cc2ccc(O)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

In an analogous manner to Example 2, pure cyclic peptide 4 was synthesized employing the Fmoc-AA-acids: Fmoc-Gly-OH, Fmoc-Nle-OH, Fmoc-Sar-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH

Scale of synthesis: 9.60 mmol (load. 0.32 mmol/g, resin 30.0 g)

Yield 41% (after purification)

Purity: 99.2% (HPLC area-%, HPLC method cf. Example 1)

Retention time: 25.9 min (HPLC method cf. Example 1)

MS (m/z): 945.5 (M+H)⁺

### Example 10

c[Gly-Tyr-Ile-Gln-Asn-Glu]-trans-4-fluoro-Pro-Leu-Gly-NH₂ (5)

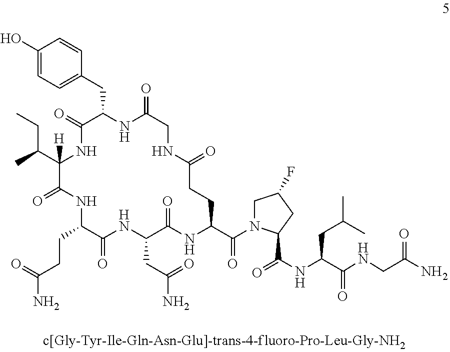 [SMILES: CC[C@H](C)[C@@H]1NC(=O)[C@H](Cc2ccc(O)cc2)NC(=O)CNC(=O)CC[C@@H](C(=O)N2C[C@H](F)C[C@H]2C(=O)N[C@@H](CC(C)C)C(=O)NCC(N)=O)NC(=O)[C@H](CC(N)=O)NC(=O)[C@H](CCC(N)=O)NC1=O]

In an analogous manner to Example 2, pure cyclic peptide 5 was synthesized employing the Fmoc-AA-acids: Fmoc-Gly-OH, Fmoc-Leu-OH, Fmoc-trans-4-fluoro-Pro-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH

Scale of synthesis: 9.60 mmol (load. 0.32 mmol/g, resin 30.0 g)

Yield: 39% (after purification)

Purity: 98.8% purity (HPLC area-%, HPLC method cf. Example 1)

Retention time: 25.7 min (HPLC method cf. Example 1)

MS (m/z): 988.5 (M+H)⁺

### Example 11

c[Gly-Tyr-Ile-Gln-Asn-Glu]-trans-4-hydroxy-Pro-Leu-Gly-NH₂ (6)

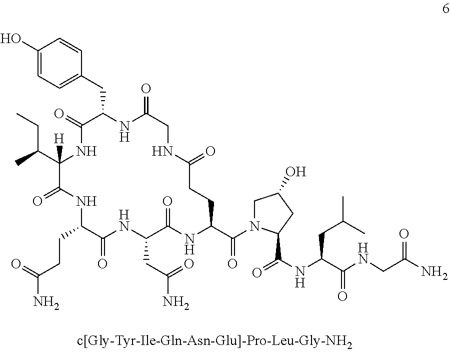 [SMILES: CC[C@H](C)[C@@H]1NC(=O)[C@H](Cc2ccc(O)cc2)NC(=O)CNC(=O)CC[C@@H](C(=O)N2C[C@H](O)C[C@H]2C(=O)N[C@@H](CC(C)C)C(=O)NCC(N)=O)NC(=O)[C@H](CC(N)=O)NC(=O)[C@H](CCC(N)=O)NC1=O]

In an analogous manner to Example 2, pure cyclic peptide 6 was synthesized employing the Fmoc-AA-acids: Fmoc-Gly-OH, Fmoc-Leu-OH, Fmoc-trans-4-tertbutoxy-Pro-OH, Fmoc-Glu(tBu)-OH, Fmoc-Asn(Trt)-OH, Fmoc-Gln(Trt)-OH, Fmoc-Ile-OH, Fmoc-Tyr(tBu)-OH, Fmoc-Gly-OH

Scale of synthesis: 9.60 mmol (load. 0.32 mmol/g, resin 30.0 g)

Yield: 22% (after purification)

Purity: 98.7% purity (HPLC area-%, HPLC method cf. Example 1)

Retention time: 23.3 min (HPLC method cf. Example 1)

MS (m/z): 987.5 (M+H)⁺.

### Example 12

### Solid Phase Cyclization

c[Gly-Tyr-Ile-Gln-Asn-Glu]-trans-4-fluoro-Pro-Leu-Gly-NH₂_{_}(5)

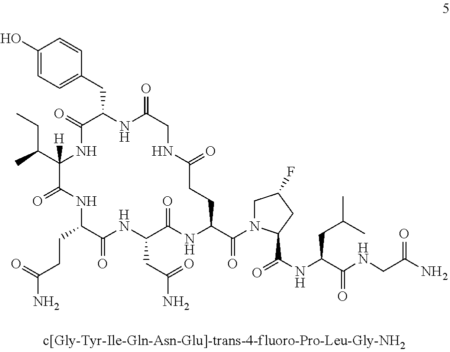 [SMILES: CC[C@H](C)[C@@H]1NC(=O)[C@H](Cc2ccc(O)cc2)NC(=O)CNC(=O)CC[C@@H](C(=O)N2C[C@H](F)C[C@H]2C(=O)N[C@@H](CC(C)C)C(=O)NCC(N)=O)NC(=O)[C@H](CC(N)=O)NC(=O)[C@H](CCC(N)=O)NC1=O]

In an analogous manner to Example 1 employing a CS536XT peptide synthesizer from CSBio, pure cyclic peptide 5 was synthesized employing the Fmoc-AA-acids: Fmoc-Gly-OH, Fmoc-Leu-OH, Fmoc-trans-4-fluoro-Pro-OH, Fmoc-Glu(OAll)-OH, Fmoc-Asn(Trt)-OH, Fmoc-Gln(Trt)-OH, Fmoc-Ile-OH, Fmoc-Tyr(tBu)-OH, Fmoc-Gly-OH. Throughout the entire synthesis, for Fmoc-cleavage 10% 4-methyl-piperidine in DMF instead of 20% piperidine in DMF was used, and all amino acid couplings in the linear sequence were conducted employing HOPy instead of HOBt. In the final PyBOP promoted cyclization on resin step, 4-methylmorpholine instead of DIPEA was used as base and the cyclization was run in DMF instead of NEP as solvent. The preparative HPLC purification of crude c[Gly-Tyr-Ile-Gln-Asn-Glu]-trans-4-fluoro-Pro-Leu-Gly-NH₂ was conducted on a Kromasil-C18-100 column (250×4.6 mm, 10 um particle size, A: 20 mM NH4OAc pH5, B: MeCN; flow: 1 mL/min; isocratic 90/10 (A/B) for 1 min, gradient from 90/10 (A/B) to 80/20 (A/B) within 1 min, gradient from 80/20 (A/B) to 75/25 (A/B) within 10 min, gradient from 75/25 (A/B) to 10/90 (A/B) within 1 min, gradient from 10/90 (A/B) to for 5 min, gradient from 10/90 (A/B) to 90/10 (A/B) within 0.1 min, isocratic 90/10 (A/B) for 6.9 min. The collected fractions were diluted with water (1:1) and concentrated/desalted by loading on a conditioned (water/ACN 90/10) Kromasil C18-100-10 column (250×4.6 mm) and eluated afterwards with water/ACN (1:1). The collected fractions (UV 280 nm, threshold 1000mAu) were rotatory evaporated to remove ACN and lyophilized afterwards to yield the pure peptide as a white lyo product

Scale of synthesis: 5.50 mmol (loading 0.55 mmol/g, resin 10.0 g)

Yield: 34% (after purification)

Purity: 98.8% purity (HPLC area-%, HPLC method cf. Example 1)

Retention time: 25.3 min (HPLC method cf. Example 1)

MS (m/z): 989.5 (M+H)⁺

### Example 13

### Solid Phase Cyclization

c[Gly-Tyr-Ile-Gln-Asn-Glu]-trans-4-fluoro-Pro-Leu-Gly-NH₂_{_}(5)

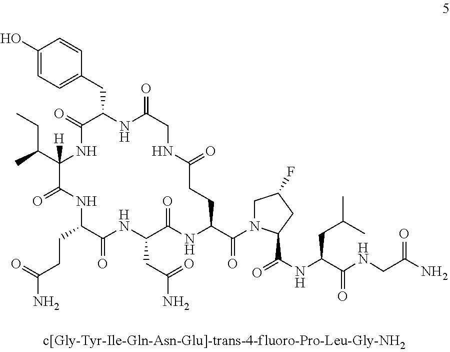 [SMILES: CC[C@H](C)[C@@H]1NC(=O)[C@H](Cc2ccc(O)cc2)NC(=O)CNC(=O)CC[C@@H](C(=O)N2C[C@H](F)C[C@H]2C(=O)N[C@@H](CC(C)C)C(=O)NCC(N)=O)NC(=O)[C@H](CC(N)=O)NC(=O)[C@H](CCC(N)=O)NC1=O]

In an analogous manner to Example 13, pure cyclic peptide 5 was synthesized employing HOBt instead of HOPy throughout the entire synthesis of the linear peptide on resin.

Scale of synthesis: 5.50 mmol (loading 0.55 mmol/g, resin 10.0 g)

Yield: 25% (after purification)

1. A process for the preparation of a compound of formula I 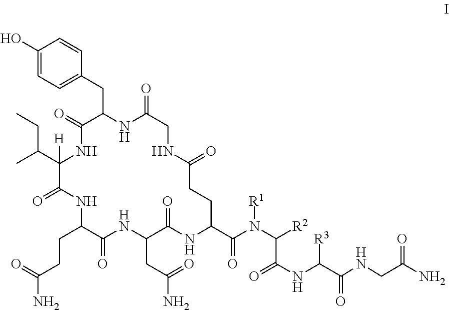
wherein
R¹ is hydrogen or C₁₋₇-alkyl and
R² is hydrogen or C₁₋₇-alkyl; or
R¹ and R² together with the nitrogen and the carbon atom to which they are attached form a 5-membered heterocyle which is optionally substituted with hydroxy or halogen; and
R³ is C₁₋₇-alkyl
or an enantiomer and/or optical isomer thereof, comprising treating a resin bound peptide precursor of the formula II 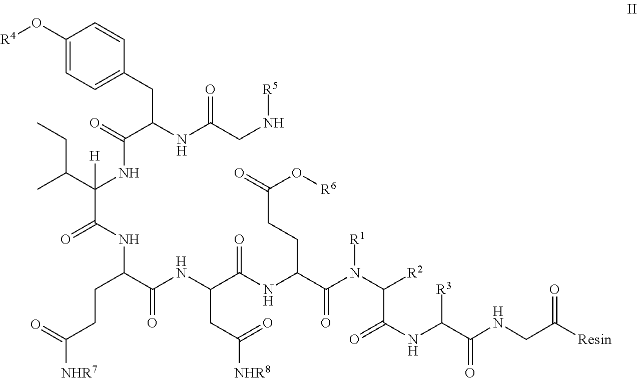
wherein
R¹, R² and R³ are as above and
R⁴ is a hydroxy protecting group;
R⁵ is Fmoc;
R⁶ is allyl, t-butyl, 1-adamantyl, 4-{N-[1-(4,4-dimethyl-2,6-dioxocyclohexylidene)-3-methylbutyl]amino}benzyl or phenylisopropyl;
R⁷ is an amide protecting group; and
R⁸ is an amide protecting group
or an enantiomer and/or optical isomer thereof,
either according to method a) or b):
a) wherein in case of R⁶ being allyl or 4-{N-[1-(4,4-dimethyl-2,6-dioxocyclohexylidene)-3-methylbutyl]amino}benzyl
a¹) the allyl group or the 4-{N-[1-(4,4-dimethyl-2,6-dioxocyclohexylidene)-3-methylbutyl]amino}benzyl group R⁶ is cleaved, in a subsequent step
a²) the Fmoc group R⁵ is cleaved, thereafter
a³) ring cyclization is effected on the resin, in a further step
a⁴) global deprotection and cleavage from the resin is effected, and optionally
a⁵) the oxytocin analogue of formula I so obtained is purified and isolated;
b) wherein in case of R⁶ being t-butyl, 1-adamantyl or phenylisopropyl;
b¹) the Fmoc group R⁵ is cleaved, thereafter
b²) global deprotection and cleavage from the resin is effected, in a further step
b³) ring cyclization is effected in solution, then optionally
b⁴) the oxytocin analogue of formula I so obtained is isolated and purified.

2. The process of claim 1, wherein the compound of formula I is further defined as a compound of formula Ia 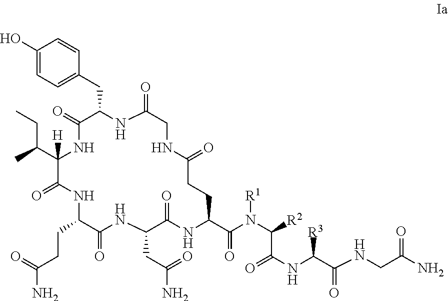
wherein R¹, R² and R³are as above and wherein the resin bound peptide precursor of formula II has the formula 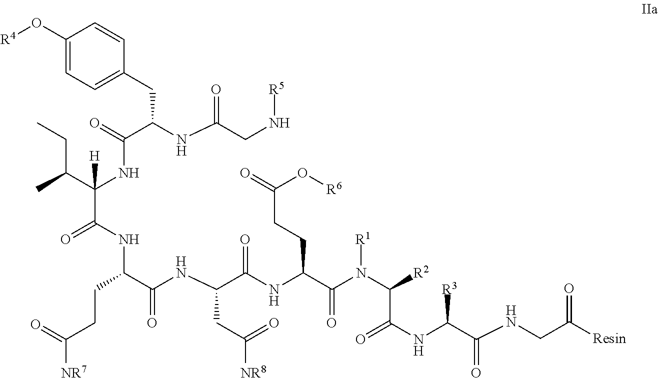
wherein R¹, R², R³, R⁴, R⁵, R⁶, R⁷ and R⁸ are as above.

3. The process of claim 1, wherein
R¹ is hydrogen or C₁₋₄-alkyl and
R² is hydrogen or C₁₋₄-alkyl; or
R¹ and R² together with the nitrogen and the carbon atom to which they are attached form a pyrrolidine ring of proline which is optionally substituted with hydroxy or halogen;
R³ stands for n-butyl or i-butyl;
R⁴ is t-butyl, allyl, trityl, 2-chlorotrityl, t-butyloxycarbonyl, t-butyldiphenylsilyl or t-butyldimethylsilyl;
R⁵ is Fmoc;
R⁶ is allyl, 1-adamantyl, 4-{N-[1-(4,4-dimethyl-2,6-dioxocyclohexylidene)-3-methylbutyl]amino}benzyl, phenylisopropyl or t-butyl;
R⁷ is trityl, 2-chlorotrityl, 4-methyltrityl; and
R⁸ is trityl, 2-chlorotrityl, 4-methyltrityl.

4. The process of claim 1, wherein
R¹ is hydrogen or methyl and
R² is hydrogen or
R¹ and R² together with the nitrogen and the carbon atom to which they are attached forms pyrrolidine ring of proline which is optionally substituted with hydroxy or fluorine;
R³ stands for n-butyl or i-butyl;
R⁴ is t-butyl;
R⁵ is Fmoc;
R⁶ is allyl;
R⁷ is trityl; and
R⁸ is trityl.

5. The process of claim 1, wherein the resin bound peptide precursor of formula II is prepared on the resin by repeated Fmoc cleavage and coupling of the respective Fmoc protected amino acids.

6. The process of claim 5, wherein the Fmoc cleavage is performed with a solution of piperidine or 4-methyl-piperidine in a suitable organic solvent.

7. The process of claim 5, wherein the Fmoc coupling is performed using a coupling agent selected from the group consisting of benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), hydroxybenzotriazole (HOBt) and N,N′-diisopropylcarbodiimide (DIC), N,N,N′,N′-tetramethyl-O-(benzotriazol-1-yl)-N,N,N′,N′-tetramethyluronium hexafluorophosphate (HBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), tetramethylfluoroformamidinium hexafluorophosphate (TFFH), 2-hydroxy-pyridine (HOPy) and 4-(4,6-dimethoxy-1,3,5-triazin-2-yl)-4-methylmorpholinium chloride (DMTMM) in the presence of an organic amine base and a suitable organic solvent.

8. The process of claim 7, wherein the resin is a 4-[(2,4-Dimethoxyphenyl)Fmoc-aminomethyl]phenoxyacetamido methyl resin.

9. The process of claim 1 wherein, in the steps
a¹) the allyl group or the 4-{N-[1-(4,4-dimethyl-2,6-dioxocyclohexylidene)-3-methylbutyl]amino}benzyl group R⁶ is cleaved in the presence of a palladium or a rhodium compound or of hydrazine;
a²) the Fmoc group R⁵ is cleaved with a solution of piperidine or 4-methyl-piperidine in a suitable organic solvent;
a³) ring cyclization is effected on the resin, using a cyclization agent selected from benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), N,N,N′,N′-tetramethyl-O-(1H-benzotriazol-1-yl)uranium hexafluorophosphate (HBTU), 1-[bis(dimethylamino)methylene]-1H-1,2,3-triazolo[4,5-b]pyridinium 3-oxid hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), 2-hydroxy-pyridine (HOPy) or 4-(4,6-dimethoxy-1,3,5-triazin-2-yl)-4-methylmorpholinium chloride (DMTMM) in the presence of an organic amine base;
a⁴) global deprotection and cleavage from the resin is effected in the presence of trifluoroacetic acid and a suitable scavenger such as thioanisole, anisole, phenol, triisopropylsilane, triethylsilane, ethanedithiol or dithiothreitol;
a⁵) optionally, the oxytocin analogue of formula I so obtained is purified and isolated.

10. The process of claim 1 wherein, in the steps
b¹) the Fmoc group R⁵ is cleaved with a solution of piperidine or 4-methyl-piperidine in a suitable organic solvent;
b²) global deprotection and cleavage from the resin is effected in the presence of trifluoroacetic acid and a suitable scavenger such as thioanisole, anisole, phenol, triisopropylsilane, triethylsilane, ethanedithiol or dithiothreitol;
b³) ring cyclization is effected in solution using a cyclization agent selected from benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), N,N,N′,N′-tetramethyl-O-(1H-benzotriazol-1-yl)uranium hexafluorophosphate (HBTU), 1-[bis(dimethylamino)methylene]-1H-1,2,3-triazolo[4,5-b]pyridinium 3-oxid hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), 2-hydroxy-pyridine (HOPy) or 4-(4,6-dimethoxy-1,3,5-triazin-2-yl)-4-methylmorpholinium chloride (DMTMM) in the presence of an organic amine base;
b⁴) optionally the oxytocin analogue of formula I so obtained is isolated and purified.

11. The process of claim 9, wherein the organic amine base is selected from pyridine, imidazole, N,N-diisopropylethyl amine, triethylamine, N-methylmorpholine, N,N-dimethyl-4-aminopyridine, 1,8-Diazabicyclo[5.4.0]undec-7-ene or 1,4-diazabicyclo[2.2.2]octane.

12. The process of claim 1, wherein the compound of formula I is further defined as a compound of formula Ia 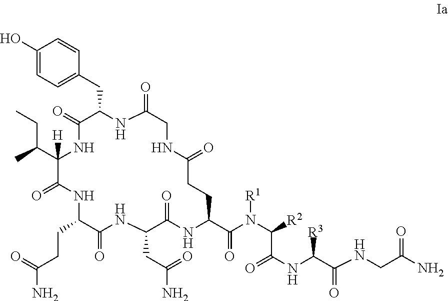
wherein
R¹ is hydrogen or C₁₋₄-alkyl and
R² is hydrogen or C₁₋₄-alkyl; or
R¹ and R² together with the nitrogen and the carbon atom to which they are attached form a pyrrolidine ring of proline which is optionally substituted with hydroxy or halogen;
R³ is n-butyl or i-butyl
or a corresponding enantiomer and/or optical isomer thereof, comprising treating a resin bound peptide precursor of formula II 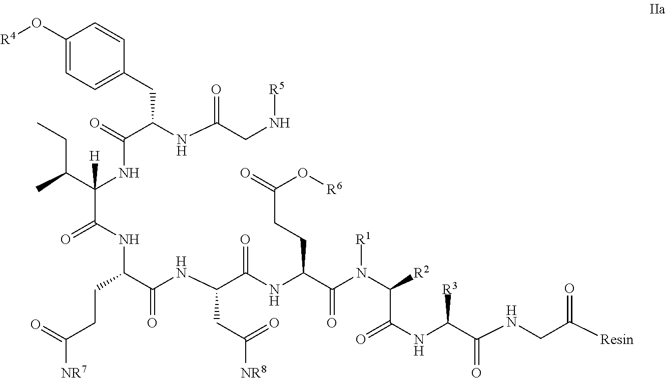
wherein
R¹, R² and R³ are as above and
R⁴ is t-butyl, allyl, trityl, 2-chlorotrityl, t-butyloxycarbonyl, t-butyldiphenylsilyl or t-butyldimethylsilyl;
R⁵ is Fmoc;
R⁶ is allyl, t-butyl, 1-adamantyl or phenylisopropyl;
R⁷ is trityl, 2-chlorotrityl, 4-methyltrityl; and
R⁸ is trityl, 2-chlorotrityl, 4-methyltrityl according to the method:
b¹) the Fmoc group R⁵ is cleaved with a solution of piperidine or 4-methyl-piperdine in a suitable organic solvent;
b²) global deprotection and cleavage from the resin is effected in the presence of trifluoroacetic acid and a suitable scavenger such as thioanisole, anisole, phenol, triisopropylsilane, triethylsilane, ethanedithiol or dithiothreitol;
b³) ring cyclization is effected in solution using a cyclization agent selected from benzotriazol-1-yl-oxytripyrrolidinophosphonium hexafluorophosphate (PyBOP), (7-azabenzotriazol-1-yloxy)tripyrrolidinophosphonium hexafluorophosphate (PyAOP), N,N,N′,N′-tetramethyl-O-(1H-benzotriazol-1-yl)uranium hexafluorophosphate (HBTU), 1-[bis(dimethylamino)methylene]-1H-1,2,3-triazolo[4,5-b]pyridinium 3-oxid hexafluorophosphate (HATU), O-(6-chlorobenzotriazol-1-yl)-N,N,N′,N′-tetramethyluronium hexafluorophosphate (HCTU), (1-cyano-2-ethoxy-2-oxoethylidenaminooxy)dimethylamino-morpholino-carbenium hexafluorophosphate (COMU), 2-hydroxy-pyridine (HOPy) or 4-(4,6-dimethoxy-1,3,5-triazin-2-yl)-4-methylmorpholinium chloride (DMTMM) in the presence of an organic amine base;
b⁴) optionally the oxytocin analogue of formula I so obtained is isolated and purified.

13. The process of claim 10, wherein the organic amine base is selected from pyridine, imidazole, N,N-diisopropylethyl amine, triethylamine, N-methylmorpholine, N,N-dimethyl-4-aminopyridine, 1,8-Diazabicyclo[5.4.0]undec-7-ene or 1,4-diazabicyclo[2.2.2]octane.
